(19) United States
(12) Patent Application Publication
(10) Pub. No.: US 2020/0069960 A1
(43) Pub. Date: Mar. 5, 2020

(54) HAIR LOSS REGROWTH AND HAIR TREATMENT DEVICE

(71) Applicant: Warren Winslow Walborn, Shelby, MI (US)

(72) Inventor: Warren Winslow Walborn, Shelby, MI (US)

(21) Appl. No.: 16/523,531

(22) Filed: Jul. 26, 2019

Related U.S. Application Data
(62) Division of application No. 15/344,962, filed on Nov. 7, 2016, now abandoned.
(60) Provisional application No. 62/251,344, filed on Nov. 5, 2015.

(51) Int. Cl. A61N 2/00; A61N 1/10; A61N 2/06

(52) U.S. Cl. CPC A61N 2/004; A61N 2/002; A61N 1/0484; A61N 2/06; A61N 1/10

(57) ABSTRACT

A hair restoration apparatus (100) restores hair loss and prevents further loss. The apparatus (100) includes a restoration insert (102). The restoration insert (102) includes a magnet substructure (104). A magnet array (106) is formed within the magnet substructure (104). The array (106) includes individual therapy magnets (108). The substructure (104) provides a base for the therapy magnets (108) to be positioned in close proximity to the scalp of a user (112).

### CROSS-REFERENCE TO RELATED APPLICATIONS

This application is based upon a division of U.S. patent application Ser. No. 15/344,962 filed Nov. 7, 2016, which is currently pending, and claims priority of U.S. Provisional Patent Application Ser. No. 62/251,344, filed on Nov. 5, 2015.

### STATEMENT REGARDING FEDERALLY SPONSORED RESEARCH OR DEVELOPMENT

Not applicable.

### REFERENCE TO A MICROFISHE APPENDIX

Not applicable.

### BACKGROUND OF THE INVENTION

### Field of the Invention

The invention relates to hair restoration devices and, more particularly, to devices implementing magnetic therapy to cure hair loss and prevent further loss.

### Background Art

Electromagnetism and its effects on the human body and other living organisms have been studied for hundreds of years. Because of the influence of electromagnetic fields on these organisms, health benefits have been ascribed to the use of magnets throughout history. For example, as early as 200 B.C., the Greek physician Galen described medical applications using “static” or “permanent” (i.e., non-time varying) magnets. During a period around 1000 A.D., a Persian physician, Ali Abbas, used magnetism to allegedly relieve “spasms” and “gout.” In more recent times, Anton Mesmer (in 1766) wrote of the benefits and anecdotal experiences of using magnets for a wide variety of ailments.

As subsequently discovered, magnetic fields are directly related to electric fields, as first shown by Hans Oersted in 1819. James Maxwell subsequently formalized this relationship between magnetic and electric forces in four equations which are now well known at the “Maxwell Equations.” One of these relationships mathematically described by Maxwell relates to the generation of an electric current within a conductor, where the conductor is moved through a static magnetic field created by a permanent magnet. This is in contrast to many historical applications of electromagnetism, which have been directed towards the use of time-varying electric fields. Health applications of magnetic fields employing permanent magnets have essentially been somewhat ignored in the medical field, and often conjure up a status in people's minds as “folk remedies” or other types of anecdotal healing aids. One example of a recently discovered use of time-varying fields relates to the use of “pulse electromagnetic fields” (PEMF's). For example, such magnetic fields are essentially used today in machines employing magnetic resonance imaging (i.e., MRI machines). Such diagnostic developments have greatly enhanced the ability of the physician to diagnose an extensive number of non-skeletal injuries and diseases without extensive invasive procedures.

Time-varying electromagnetic fields have recently been applied to issues associated with hair loss. Today, hair loss in the form of male pattern baldness effects over 40 million men in the United States alone. Male pattern baldness is the most common type of hair loss in men. The baldness history path usually follows a typical pattern of receding hairline and hair thinning on the crown. It is thought that such hair loss and receding hairline is caused by hormones and genetic predisposition. Male pattern baldness does not necessarily indicate any type of medical disorder. However, as is well-known, such baldness can substantively effect self-esteem or otherwise cause anxiety or other emotional problems. Unfortunately, such hair loss or male pattern baldness is usually permanent. That is, there is no known prevention for male pattern baldness. The medical name for male pattern baldness is androgenetic alopecia.

Returning to the concepts associated with post-electromagnetic fields, research has been directed to the utilization of such magnetic fields for the prevention of hair loss and stimulation of hair regrowth. One such process is commonly known as electrotrichogenesis (ETG). A discussion regarding the use of ETG is found in Maddin, et al, “The Biological Effects of a Pulsed Electrostatic Field with Specific Reference to Hair: Electrotrichogenesis,” University of British Columbia, Division of Dermatology, July-August 1990, Volume 29, No. 6. ETG clinical trials were conducted at the University of British Columbia in 1989, and again in 1992. The results of the trials are considered relatively successful, and are discussed in Maddin, et al, “Electrotrichogenesis: Further Evidence of Efficacy and Safety on Extended Use,” International Journal of Dermatology, Volume 31, No. 12, December 1992.

The principal researchers associated with the ETG trials have acknowledged that the exact beneficial mechanism is unclear. The researchers suggest that dormant follicle cells groups are similar to the fibroblast cell groups identified by Becker and Selden, and are thus capable of regeneration.

Beside the use of pulsed electromagnetic fields, much of the recent research associated with reduction of hair loss and hair regrowth has been directed to chemical solutions. For example, one relatively well-known drug is Minoxidil, a topical ointment applied to the affected areas of hair loss. With respect to Minoxidil and other drugs currently on the market for stopping hair loss and facilitating hair regrowth, most of these drugs are relatively costly and time consuming to use. Other forms of treatment for hair loss have been commercially available for some time, and include hair transplants, wigs or hairpieces, and even paint and tattoos are often used.

With respect to the physicality of hair growth, each hair sits in a cavity in the skin referred to as a follicle. Baldness in men occurs when the follicle shrinks over time, resulting in shorter and relatively finer hair. The end result is a relatively small follicle, with no hair inside. Ordinarily, hair should grow back. However, in men who are balding, the follicle fails to grow a new hair. The exact reasons for this failure of new hair growth is not particularly well understood by researchers, although it is clear that the baldness occurs as a result of genetic makeup and male sex hormones. Of particular importance, even though the follicles grow relatively small, they still remain alive. This continuous life suggests the possibility of new growth.

Although much of recent development relating to hair loss and hair regrowth has been associated with chemical solutions, there has been continuing work with regard to the use of static magnetic fields associated with permanent magnets.

Turning to specific references, Knox, et al, U.S. Pat. No. 6,267,720 issued Jul. 31, 2001, is directed specifically to a system and method for hair loss reduction and regrowth. The Knox, et al, device is a form of removable headwear which is provided for therapeutically applying a permanent magnetic field to hair follicles located in the scalp, so as to retard hair loss and promote hair regrowth. The flexible sheet of magnetic material having a series of alternating north-south poles is placed with a head covering that, in turn, is placed upon the head of a subject. The head covering, which may be suitable for nighttime wear, may not be deemed socially acceptable for wear during the day. To enable maximization of the exposure of the scalp to the magnetic fields, the head covering is configured so as to permit an item of conventional headwear to be placed over and cover the inner head covering. More specifically, and with reference to the numbers in the drawings and the specification of Knox, et al, a magnetic sheet 12 is received within an inner cap 16. The magnetic sheet 12 is described as being attached to a lining of an inner cap 16 or, preferably, is placed within an inner sleeve 18 formed in an inner lining of the inner cap 16. With such placement, the portion of the inner cap lining underlying the inner sleeve 18 forms a treatment surface 20. The magnetic sheet 12 is received by and held within the inner cap 16 so as to obtain proper positioning of the magnetic fields associated with the sheet 12.

The inner cap 16 is also provided with an adjustable headband 22, so as to provide use over a variety of different head sizes. A hook and loop fastening system is disclosed for providing such adjustability, consisting of a loop section 24 formed in an outer surface of the headband 22, with a hook section 26 located on an adjustment band 28.

An outer cap 32 may be placed over the inner cap 16, where the appearance of the inner cap 16 is not deemed to be appropriate. The style of the outer cap 32 can be of any conventional cap or hat design, with a baseball-type design shown in the Knox, et al, drawings. The purpose of the outer cap 32 is one of concealment, relative to the inner cap 16.

The magnetic sheet 12 consists of a series of magnetic fields, having a particular alignment of magnetic polarity. Specifically, the adjacent areas of the magnetic sheet 12 are of opposite magnetic polarity. An example of one commercially available type of magnetic sheet 12 is described as being fabricated out of a material identified as ELASTOMAG™ manufactured by Nikken, of Irvine Calif. Knox, et al, continues to describe the concept that the magnetic sheet will, for most instances, measure 3″ by 4⅛″. It will also have a 1½″ thickness. The inner cap 16 is disclosed as being a multi-layer mesh cap, such as a custom made pro-style polymesh seamed front offered by Headmaster, Inc. of Santa Ana, Calif.

Other concepts regarding devices for hair regrowth methods and apparatus also exist in recent history. For example, Groux, et al, U.S. Pat. No. 5,251,623 describes the concept of attaching a low voltage positive or negative polarity signal having a selective frequency to electrodes which are maintained closely proximate to a subject's scalp. The subject undergoes a hair regrowth treatment cycle spanning a period of about 32 weeks, during which one or two of the treatments are applied per week. Groux defines a particular treatment schedule, claiming that two treatments per week for a period of 32 weeks should be sufficient.

A further patent directed to concepts associated with hair regrowth is Anderson, U.S. Pat. No. 7,819,828 issued Oct. 26, 2010. The Anderson patent discloses a device 10 having a rolling scalp activator 20. The purpose of the scalp activator 20 is to roll over a person's scalp. The device 10 includes a spindle 40 and a spinning handle 50. A motor 65 allows the activator 20 to roll without the exertion of any manual forces. A motor 65 allows the activator 20 to roll without the exertion of any manual forces. FIG. 4 illustrates a head covering 300 having one of the devices attached to the covering by a removable attachment 320. The attachment can include a Velcro or removable adhesive. The device has projections 330 that rub against a person's scalp when the covering 300 is placed against the scalp. The device is disclosed as providing for increase of capillary growth to the scalp.

Another patent uncovered during a prior patent search is Pinna, U.S. Pat. No. 5,595,564 issued Jan. 21, 1997. The Pinna patent is directed to a device for retarding hair loss and for stimulating regrowth which comprises an electrical pulse generator connected to an electrically conducting cable extending within a shaped structure to be applied to a person's skull. With reference to the numerals in the drawings and disclosure of Pinna, a support element 1 is associated with a series of electromagnetic wave generator elements 2, which are connected to a regulator member 3 and a powering member 4. The support element 1 is in the form of a helmet or hat, with two stiffening parts 1A and 1B. The hat 1 is formed of plastics material or fabric, and includes a plurality of seats for housing and retaining the generators. These latter consist for example of a cable arranged in a “daisy” loop. The powering member 4 can be a commercially available battery or plug for connecting the device to an electronic main. Depending on the type of power supply and generators 2, the regulator member 3 is arranged so as to feed the generators with power and generate waves having an intensity of at least one gauss, in proximity to the generators.

The regulator member 3 also has a conventional interface 3A by which a user can set a desired frequency and intensity. The user can also choose the type of electromagnetic wave, which can, for example, be sinusoidal or semisinusoidal, trapezoidal, triangular, periodic or inter trains of adjustable length and frequency, or in waves which alternate as adjustable frequency.

Pinna further describes a concept that the regulator member 3 and the powering member 4 could advantageously be combined into a single component. The regulator member could also comprise a programmable electronic circuit, preferably a microprocessor for powering the generators 2 so as to generate a periodic sequence of electromagnetic waves of preset variable intensity, frequency and type. In this manner, it would be possible to generate, for a set period of, for example, thirty minutes, an electromagnetic wave sequence of the following type: for the first ten minutes continuous sinusoidal waves at 50 hertz and 4 gauss; for the next ten minutes triangular waves at 40 hertz and 3 gauss lasting one minute, followed by a 30 second pause; and for the last ten minutes waves as for the first ten minutes. Advantageously the circuit would be programmed by the device manufacturer, so that a user would only be able to turn the device on and off. Pinna explains that he has experimentally found that the useful frequency and intensity of the electromagnetic waves generated by the generators range from 10 to 100 hertz, and from 1 to 100 gauss respectively.

Other devices utilizing magnetic fields for various physiological applications include for example, a magnetic stimulator having a skullcap-shaped coil as disclosed in Cadwell, U.S. Pat. No. 5,116,304 issued May 26, 1992. The Cadwell patent discloses a magnetic stimulator coil 13 which is connected to a power supply 15. The coil 13 is wound from the bottom to the top, such that its shape defines a skullcap. That is, the shape is a somewhat flat truncated cone with sides that curve slightly outwardly. While flexible enough to be coiled, the coil wire 21 has a relatively large AC carrying capacity for its size. The preferred wire is litz wire, or copper strip wire. Such wire has a relatively large periphery/cross-sectional area ratio. A layer of soft material 23 is located on the inside of the coil 13, so as to provide a cushion between the coil and a human cranium 17 positioned beneath the coil 13. When triggered, the power supply produces sinusoidally fluctuating electrical power. The power is adequate so as to create a magnetic field suitable for stimulating deeply located neurons 17 of the human cranium 17, positioned beneath the coil 13. The magnetic fields generated are considered adequate so as to create a neuron depolarizing electric field deeply within the cranium.

Other devises employ magnets and are directed to use around the skull, but are not necessarily associated with manipulation of internal organs or cells of the human being. For example, Kronenberger, U.S. Pat. No. 7,140,047 issued Nov. 28, 2006, describes the combination of a headwear piece, an accessory and a series of discrete elements. The headwear piece includes a crown which defines an opening through which a wearer's head can project with the headwear piece in an operative position on the wearer's head. The crown has an inside surface with a portion for frictionally engaging a wearer's head with the headwear piece in the operative position and a first layer having an exposed outer surface. The accessory and the series of discrete elements are magnetically attracted to each other through the first layer, so as to releasably maintain the accessory in a stored state on the headwear piece. The series of discrete elements include at least two discrete elements that are adjacent to each other so as to cooperatively cause a localized magnetic attractive force to be generated between the accessory and the first and second discrete elements.

Other references are directed to devices employing magnetic field therapy, but are not necessarily directed to headgear. For example, Kraus, U.S. Pat. No. 5,415,617 issued May 16, 1995, discloses an applicator coil for an electromagnetic therapy apparatus. The coil has a form of its own of a sphere deformed into an oval and which is suited to rendering a low frequency magnetic alternating field. This alternating field is operative on projecting parts of a human or animal body, such as the shoulder region or the face of a person.

More specifically, the applicator coil includes a deformed multi-layer wire winding 10, having turns which are substantially egg-shaped or have the form of trapezia with rounded ends. The winding density can be somewhat greater at the wider end than at the narrower end of the coil. The winding 10 is enclosed in a plastics casing 12, which consists of two shells 12a and 12b having flange-like edges 15, 16, which are bonded or otherwise welded tougher. At the narrower or sharper end of the outer shell 12a, a handle 14 is fitted. The applicator coil has a narrower opening 18, bounded by a rounded part of the outer shell 12a. At the wider end 20, the applicator coil is substantially flat up to the flange 15. The coil winding 10 is provided with terminals 21, which are fitted in a handle 14 and connected to a connecting cable 22. Through the connecting cable 22, the applicator coil can be connected to a generator unit for generating a low frequency, sinusoidal alternating current. The frequency is disclosed as being variable between 2 and 20 hertz.

In the '617 patent, the applicator coil is provided with a device for feeding a treatment fluid. Openings or nozzles 24 are formed through the inner shell 12b, which are connected by means of branch lines 26 to a feed line 38. The feed line 38 is then further connected to a hose 30. The hose 30 is connected to a source of a fluid or gaseous treatment medium during use of the applicator coil, and permits the magnetic field treatment to be assisted by an application of a suitable fluid. The fluid is disclosed as being a healing medium or cosmetic agent.

Electrodes can be provided on the inside of the casing 12, with which an electric field can be caused to act on the body part to be treated, either galvanically or capacitively. The electrodes can be capable of connection through their own lead to a suitable generator 31, for an alternating voltage which is either symmetrical or asymmetrical relative to zero or a unipolar voltage with low frequency fluctuations, such as a DC voltage on which an alternating voltage is superimposed. Alternatively, connection can be made from their own receiver coil 36 to lines 32, 34, in which a corresponding low frequency voltage is induced through the winding 10. The receiver coil can be rotatably arranged in the plastics casing 12 through use of, for example, a pin 36a, in order to be able to adjust the intensity and/or phase angle of the induced alternating voltage and be coupled to a rectifier circuit, if desired. This provides for generating an alternating voltage asymmetrical with respect to zero, or a fluctuating unipolar voltage for the electrodes.

Woo, U.S. Pat. No. 6,379,295 issued Apr. 30, 2002, is directed to a method and apparatus for alleviating or curing human afflictions, ailments and diseases by application of magnetism. In use, the Woo patent discloses the concept of dividing designated treatment areas of the head into six treating regions, identified by letters A, B, etc. Magnets are attached directly to the head in the designated areas. The magnets may be applied to the entire area of the head concurrently, or partially by region at a time. The regions of the head are disclosed as being treated separately, one at a time, at intervals of ten hours, with an equal amount of treatment time. First, the magnet or magnets are applied to the front part, then the top part, with an equal amount of treatment time for each. At intervals of ten hours, the lower back part is treated first, then the upper back part is treated. At intervals often hours, side parts are also treated consecutively, with an equal amount of treatment for each. The regions of the head are disclosed as having a total size of approximately 160 square inches, defined from an eyebrow line through a head median line, to an occipital hairline. At least one magnet is utilized, with the magnet having a size of 0.25″ by 0.25″, to 10″ by 16″. The magnet is to be maintained in contact with at least one of the treating regions of the head for a period of 15 to 180 minutes to treat afflictions. The total flux of the magnet applied to the treating regions of the head is in a range of 30-250,000 gauss.

Lopez, et al, U.S. Pat. No. 5,720,046 issued Feb. 24, 1998, is directed to articles of humanwear having magnetic structures for producing healing effects. The '046 patent describes various types of merchandise, including torso-worn garments, gloves and gear for the human head and body appendages. All of these merchandise employ a magnetic structure which corresponds to selected acupressure regions and points on the human torso, hands, head and body appendages for affecting therapeutic magnetic exposure, when worn by a user. In FIG. 16a of the '046 patent, human headwear merchandise is shown in the form of a headband with an “across-the-head” band design having an imbedded magnetic structural arrangement in the form of discrete permanent magnets having a first or second polarity positioned inwardly toward a user's head. The magnets are positioned at the headband's side and cross member band portions. FIG. 16b shows a rear view of the apparatus. FIG. 16a and discloses additional discreet permanent magnets which also have a first polarity positioned inwardly toward a user's head, and spaced apart substantially at the rear of the headband. Correspondently, FIG. 16c discloses a modified version of the headwear illustrated in FIGS. 16a and 16b, and expressly discloses a visor provided for purposes of encouraging outdoor use of the headgear.

Various references are specifically directed to the structure, material content and/or overall shape of the magnets and the medium on which magnets may be applied. For example, Ardizzone, U.S. Pat. No. 5,871,438 issued Feb. 16, 1999, discloses a flexible magnetic pad which is provided with constantly alternating polarity zones. The alternating polarity zones are disclosed as therapeutically effecting blood vessels so as to increase blood flow, irrespective of the orientation of any blood vessel with respect to the pad. The polarity zones are arranged in an alternating pattern throughout the pad.

Baermann, U.S. Pat. No. 4,549,532 issued Oct. 29, 1985, and was the subject of Reexamination Certificate B1 4,549,532 issued Aug. 11, 1998. Baermann discloses a flexible magnetic sheet for therapeutic use. The sheet is constructed of a rubbery-flexible synthetic material in which permanent-magnetic ferrite particles have been embedded. The surface of the sheet which faces the body side to be treated is magnetized with magnetic poles of alternating polarities. The poles are in the form of a geometrical shape, such as concentrically arranged rings, sectors, quadrangles and the like.

Mitsuno, et al, U.S. Pat. No. 5,304,111 issued Apr. 19, 1984, discloses a flexible planar magnetic sheet for therapeutic use. The sheet has a regular repeating pattern of curved first and second areas of alternating magnetic polarity, arranged so that a line traverses a series of the curved areas in a majority of possible orientations of the line along the surface plane of the sheet, with respect to the pattern. One embodiment of Mitsuno, et al, discloses the pattern as having an array of polka dot regions of one polarity uniformly sized and spaced on a background of opposite polarity. As an alternative, the pattern is disclosed as being capable of being shaped as a series of interdigitated undulating first and second areas of alternating magnetic polarity. The areas each have curved boundaries which are defined by wavy lines, and a width which varies between a maximum and a minimum proceeding generally lengthwise along the wavy lines of a lengthwise periodicity, so as to define lobe-shaped portions of each area.

A still further reference utilizing a magnetic field is disclosed in Schurig, et al, U.S. Pat. No. 5,092,835 issued Mar. 3, 1992. The Schurig, et al, patent discloses an apparatus and method which allegedly supplies healing power to the brain and nervous system. The method includes applying a constant magnetic field within a cap or strap arrangement to the head or nervous system of a subject. The nerves are stimulated with electric signals applied to neural pathways to the brain, located in the hands, feet and other parts of the body. The '835 patent is expressly directly to use in aiding individuals suffering from nervous system disorders, including disorders such as pervasive developmental disorder, autism, disorders caused by hypoxia, trauma and drugs. The treatment can also be utilized for some forms of schizophrenia, mild learning disabilities disorders and also to heighten the mental and physical abilities of people not having any particular afflictions.

### SUMMARY OF THE INVENTION

In accordance with the invention, an apparatus is provided which is positioned on or adjacent to the scalp of a user, with the use of magnet therapy for purposes of curing hair loss of any type, and also for preventing further hair loss. The apparatus can be in the form of a hat insert or similar solution which enables magnetic therapy magnets to remain in close proximity to the affected scalp areas for extended periods of time.

### BRIEF DESCRIPTION OF THE SEVERAL VIEWS OF THE DRAWING

The invention will now be described with reference to the drawings, in which:

FIG. 1 is an exploded view of an illustrative embodiment of the invention, illustrating an embodiment of the hair restoration apparatus with an example head covering in the form of a billed cap, and a restoration insert which is positionable within the cap, and further showing a portion of the restoration insert in an enlarged view, which illustrates several of the therapy magnets as they are embedded within a magnet substructure;

FIG. 2 is a perspective view of a user wearing the billed cap and hair restoration apparatus illustrated in FIG. 1, in a fully assembled state;

FIG. 3 is a side-elevation view of the restoration insert illustrated in FIG. 1, with the restoration insert being shown in a stand-alone configuration;

FIG. 4 is a side-elevation view of a user, with the user wearing a night cap and showing the use of the restoration insert in a cut-out configuration as positioned under the night cap and adjacent the scalp of the user;

FIG. 5 is a partially exploded view showing the relative positioning of the restoration insert as it can be inserted into a commercially available Stetson hat;

FIG. 6 is a partially exploded view similar to FIG. 5, but showing the restoration insert in a position so as to be inserted into the interior of a western, full-brim hat;

FIG. 7 is a partially exploded view similar to FIGS. 5 and 6, and showing the restoration insert in a position so as to be inserted into a head scarf having a relatively long tie-back;

FIG. 8 is a partially exploded view similar to FIG. 7, and showing the restoration insert in a position so as to be inserted into a head scarf having a relatively short tie-back;

FIG. 9 is a view of an alternative embodiment of a hair restoration apparatus in accordance with the invention, with the apparatus utilizing an active pulsed electrostatic field applied to the scalp through the use of a pulse generator and electrodes placed at various locations on a skull cap to be worn by the user; and

FIG. 10 illustrates the alternative embodiment of a hair restoration apparatus in accordance with the invention as illustrated in FIG. 9, but showing the restoration apparatus as worn by a user with a skull cap.

### DETAILED DESCRIPTION OF THE INVENTION

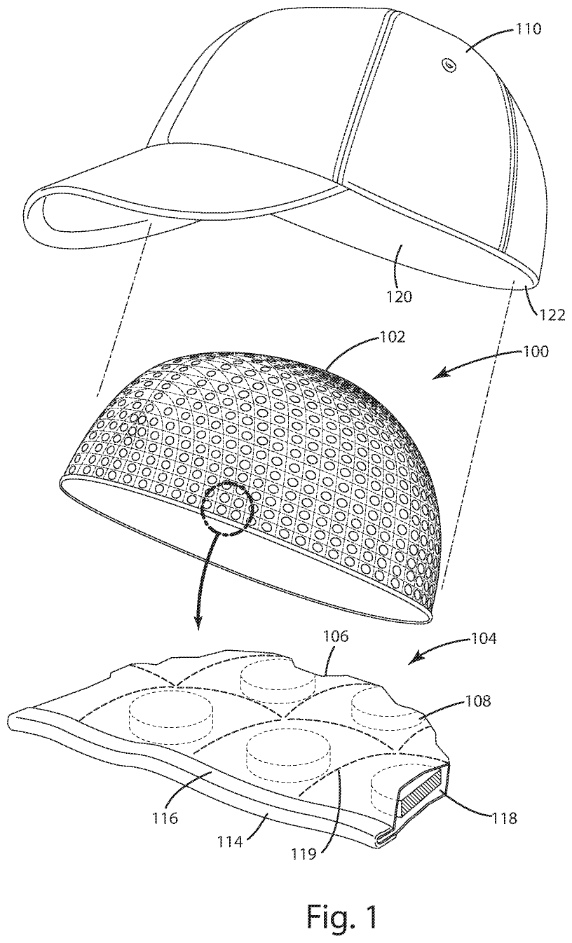

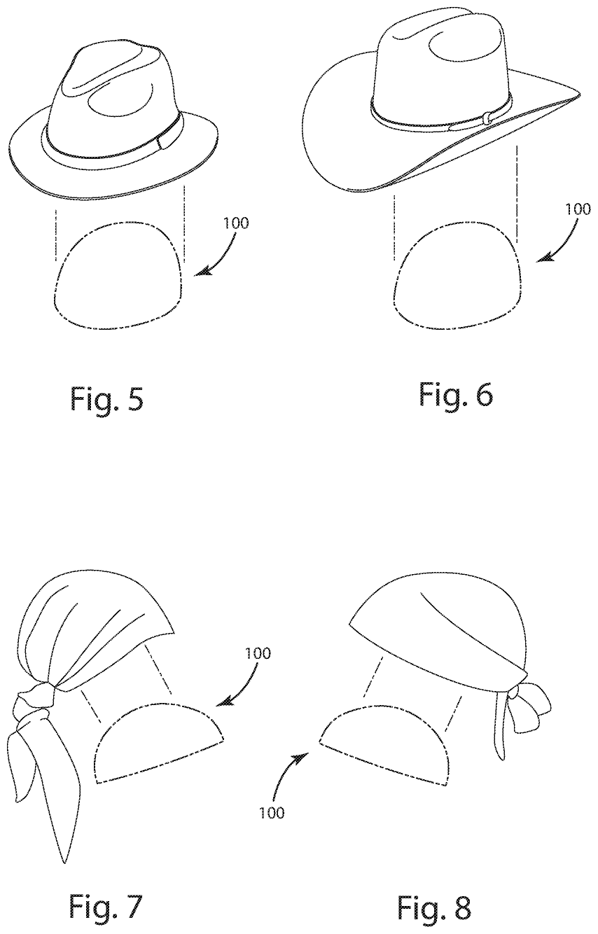

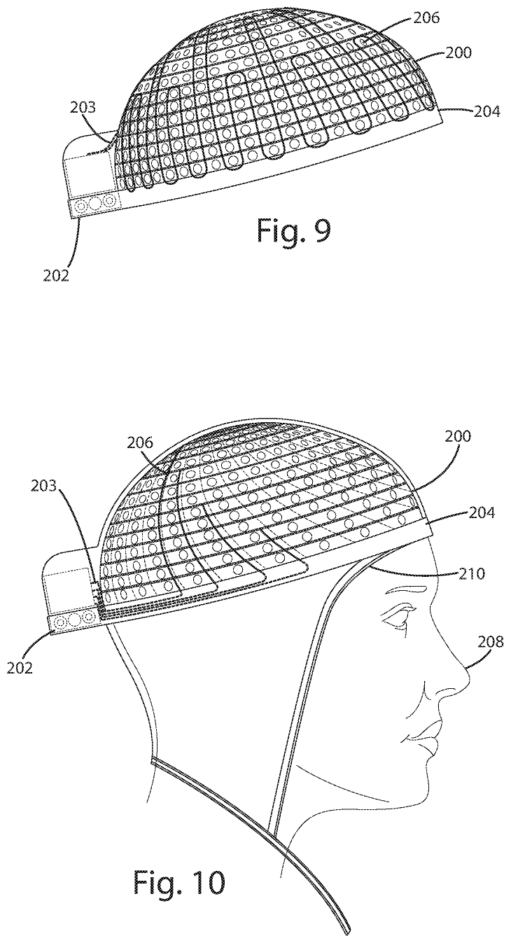

The principles of the invention are disclosed, by way of example, in a hair restoration apparatus 100 as illustrated in FIGS. 1-8. Still further, other principles of the invention are disclosed, by way of example, in an alternative embodiment of a hair restoration apparatus 200 as illustrated in FIGS. 9 and 10. The hair restoration apparatus 100 in accordance with the invention provides for the application of magnetic therapy to a user's scalp, for purposes of restoring hair loss and the prevention of further loss. The hair restoration apparatus 100 in accordance with the invention further provides for the use of a restoration insert having a magnet substructure and magnet array for implementing the magnetic therapy. Further, the apparatus 100 in accordance with the invention is structured so as to be adaptable to various types of headgear for purposes of allowing use of the hair restoration apparatus 100 in a hidden configuration.

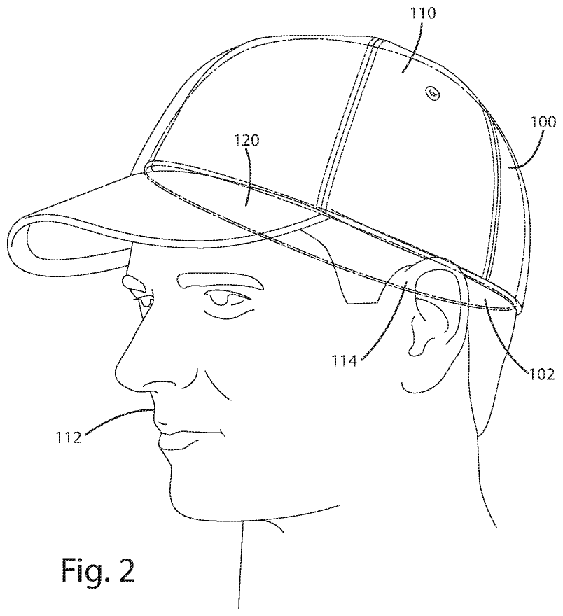

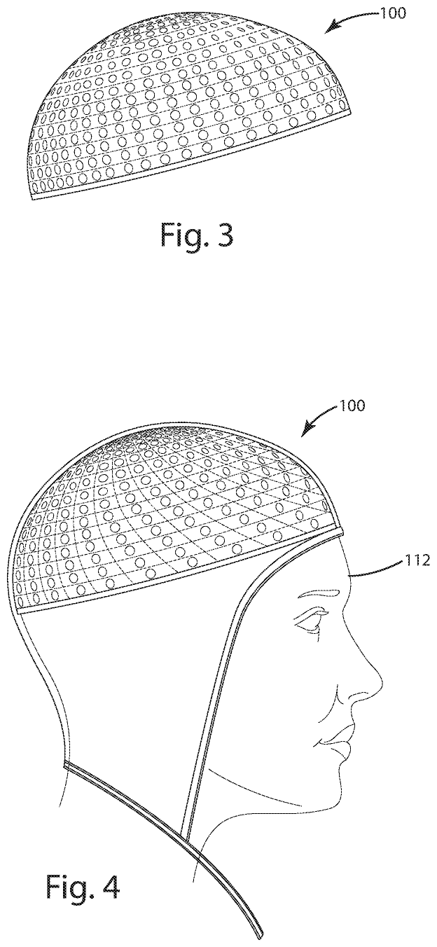

Turing to the specific drawings, the hair restoration apparatus 100 is illustrated in a partially exploded view in FIG. 1. Also, FIG. 3 illustrates a side-elevation view of the restoration apparatus 100. With reference first to FIG. 1, the restoration apparatus is illustrated as comprising a restoration insert 102. The restoration insert 102, as illustrated in FIG. 1, includes a magnet substructure 104. A section of the magnet substructure 104 is illustrated in a partially enlarged view in FIG. 1. The magnet substructure 104 has a dome-like configuration. A magnet array 106 is formed on or within the magnet substructure 104. The magnet array 106 comprises a series of individual therapy magnets 108 are positioned on or within the magnet substructure 104. The purpose for the magnet substructure 104 is to provide a base for the therapy magnets 108 which will position the therapy magnets 108 in close proximity to the scalp of a user 112 (see FIG. 2).

With respect to the magnet array 106 and individual therapy magnets 108, the magnets 108 can be coupled to the magnet substructure 104 through the use of a number of structures and processes. For example, as illustrated in FIG. 1, the magnet substructure 104 can comprise a fabric structure which may be multi-layered. That is, the magnet substructure 104 may include a lower fabric layer 114 and an upper fabric layer 116. The fabric layers 114, 116 could be stitched or otherwise sewn together along a stitch line 119 as shown in FIG. 1. Further, the fabric layers 114, 116 can be formed with pockets 118 positioned between the lower and upper fabric layers 114, 116, respectively. Each of these pockets can include one of the therapy magnets 108, again primarily shown in FIG. 1.

With the restoration insert 102 formed with the magnet substructure 104 and magnet array 106, the insert can be positioned on the head and scalp of a user 112 as illustrated in FIG. 2. With the restoration insert 102 positioned on the scalp of the user 112, the lower fabric layer 114 will abut the scalp of the user 112.

If desired, the restoration insert 102 can also be worn between the scalp of the user 112 and a desired head piece, such as the billed cap 110 illustrated in FIGS. 1 and 2. If desired, the restoration insert 102 can be maintained as a separate and independent element, relative to the billed cap 110. As an alternative, and one which may be a preferable configuration, the restoration insert 102 may be permanently or releasably coupled to the interior 120 of the billed cap 110. Securing the restoration insert 102 to the interior 120 of the billed cap 110 may provide additional convenience for the user 112, in addition to avoiding potential slippage of the restoration insert 102 from its optimal position on the scalp of the user 112.

One issue which can arise relates to the size of the user's head and the appropriate scalp area to be covered by the restoration insert 102. Because a person's scalp area and head size can vary relative to others, the overall size of the restoration insert 102 can be varied and “custom-fitted” for an individual user 112. Alternatively, the magnet substructure 104 (which is shown as comprising lower and upper fabric layers 114, 116, respectively in FIG. 1) can be constructed of a somewhat stretchable material, and may also include a headband 122 which consists of an appropriate elasticity so as to be adjustable with respect to its perimeter length.

In accordance with the foregoing, and as shown in FIGS. 1 and 2, the hair restoration apparatus 100 utilizes magnet therapies for purposes of curing hair loss of any type, and to prevent further hair loss. It is believed that magnets such as the therapy magnets 108, if positioned appropriately adjacent to the user's scalp, will improve blood flow to underlying tissues. It should be noted that this concept is also used with respect to curing and prevention of diseases and other medical problems which may be associated with various parts of a patient's body. To appreciate the application of magnet therapy for curing hair loss and preventing further hair loss, concepts associated with magnetism should be understood. As is known, a “magnet” is a material or object which produces a magnetic field. The magnetic field is invisible but is responsible for the most notable property of a magnet; that is, a force which pulls on other ferro magnetic materials, such as iron, and attracts or repels other magnets.

A “permanent magnet” is an object made from a material that is magnetized and creates its own persistent magnetic field. Such a magnet can be as simple as a refrigerator magnet utilized to hold notes on a refrigerator door. Any materials that can be magnetized or ones which are strongly attracted to a magnet, are typically called ferro magnetic. Such permanent magnets include materials formed of iron, nickel, cobalt, some alloys of rare earth materials, and some naturally occurring minerals, such as lodestone. In fact, all substances respond, at least weakly, to a magnetic field.

The overall strength of a magnet is measured by its magnetic moment or, alternatively, the total magnetic flux produced by the magnet. The local strength of magnetism in a material is measured by its magnetization.

With respect to terminology associated with the strength and range of a magnet, a “magnetic field” is typically defined as a mathematical description of the magnetic influence of electric currents and magnetic materials. A magnetic field at any given point is specified by both a direction and a magnitude, thereby being a vector field. Such a magnetic field is most commonly defined in terms of the Lorentz Force which it exerts on moving electric charges. The magnetic field is often merely referred to as the “magnetic flux density”. To physicists, this flux tendency is also known as the magnetic B field.

In the same regard, the magnetic moment [also referred to as a magnetic dipole moment) is a vector which characterizes a magnet's overall magnetic properties. A magnet produces both its own magnetic field and response to other magnetic fields. The strength of the magnetic field produced is at any given point proportional to the magnitude of its magnetic moment. Also, when the magnet is put into an external magnetic field, produced by a different source, the magnet is subject to a torque tending to orient the magnetic moment parallel to the field. The amount of this torque is proportional both to the magnetic moment and the external field. A magnet can also be subject to a force driving it in one direction or another, according to positions and orientations as a magnet and a source.

The magnetic field B, or the “magnetic flux density,” is also sometimes referred to as the “magnetic induction.” The gauss is typically abbreviated as “G” and is measured in “cgs” (centimeter-gram-second) units. One gauss is typically defined as corresponding to 1 maxwell per square centimeter. One gauss is also equal to 10⁻⁴ tesla.

An alternative embodiment of a hair restoration apparatus in accordance with the invention is illustrated in FIGS. 9 and 10, and identified as hair restoration apparatus 200. The hair restoration apparatus 200 includes a skull cap 204 which is shaped in the form of the head of a user 208. However, unlike the previously described embodiment of a hair restoration apparatus in accordance with the invention, the apparatus 200 includes a pulse generator 202 to produce electrical pulse waves at predetermined intervals, which can be varied by the user. The pulse generator 202 is adapted to apply the pulse signals to a series of electrodes 206 positioned at various spaced apart intervals on the skull cap 204. The pulse generator 202 specifically applies the pulse to electrostatic signals applied through electrodes 206 so as to generate electromagnetic energy which is then applied to the scalp of the user 208.

FIG. 9 illustrates the hair restoration apparatus embodiment 200 in a stand alone configuration. FIG. 10 illustrates the hair restoration apparatus 200 as worn by the user 208. The apparatus 200 as shown in FIG. 10 is also used with a covering cap or similar device 210 utilized to cover the head of the user 208 when used. Pulse generators such as the pulse generator 202 are commercially available, as well as electrodes which can correspond to the electrodes 206. Further, the strengths and frequency of the pulses generated by the pulse generator 202 can typically be varied by the user 208.

For purposes of construction, the electronic or electric pulse generator 202 can be connected to an electrical conducting cable 203 which is connected to the pulse generator 202 and a source of electrical power as well as connections between the pulse generator 202 and electrodes 206. It should further be noted that the pulse generator 202 is utilized to provide a pulsed electrostatic field between an interior of the head covering 210 and the scalp of the user 208.

As an example of experimental testing of the apparatus in accordance with the invention, the pulse generator 202 can be utilized to provide a pulsating electromagnetic field around the cable 203 with an intensity of at least 1 Gauss in proximity to the cable 203. In addition, the sizing and configuration of the hair restoration apparatus can be such that intensity of the electromagnetic field generated by the pulse generator 202 is substantially negligible at 5 millimeters from a surface of the conducting cable 203.

Still further, the head covering 210 and the hair restoration apparatus 200 can utilize an electrical pulse which comprises a duration of approximately 2 milliseconds, with a frequency of generation of approximately 50 pulses per second.

It will be apparent to those skilled in the pertinent arts that other embodiments in accordance with the invention can be designed. That is, the principles in accordance with the invention are not limited to the specific embodiments described herein. Accordingly, it will be apparent to those skilled in the art that modifications and other variations of the above-described illustrative embodiments of the invention may be effected without departing from the spirit and scope of the novel concept of the invention.

1. In combination, a head covering and hair restoration apparatus adapted for use for implementing magnetic therapy for curing hair loss and preventing further hair loss, said hair restoration apparatus comprising:
a restoration insert for enabling magnetic therapy and positionable between a scalp of a user and said head covering; and
said restoration insert having a magnetic substructure, said magnetic substructure providing for the enablement of therapy magnets to remain in close proximity to affected scalp areas of said user for extended periods of time.

2. A head covering and hair restoration apparatus in accordance with claim 1, characterized in that said restoration apparatus comprises a magnet array formed on or within said magnetic substructure; and
said magnetic substructure with said magnet array forming a magnetic field resulting in a pattern of alternating magnetic polarities extending substantially across a surface of said magnetic substructure formed by said magnet array.

3. A head covering and hair restoration apparatus in accordance with claim 2, characterized in that said magnet array comprises a plurality of individual therapy magnets.

4. A head covering and hair restoration apparatus in accordance with claim 3, characterized in that said individual therapy magnets are permanent magnets.

5. A head covering and hair restoration apparatus in accordance with claim 4, characterized in that said magnetic substructure comprises a fabric structure.

6. A head covering and hair restoration apparatus in accordance with claim 5, characterized in that said fabric structure is multi-layered.

7. A head covering and hair restoration apparatus in accordance with claim 6, characterized in that said multi-layered fabric structure comprises a lower fabric layer and an upper fabric layer.

8. A head covering and hair restoration apparatus in accordance with claim 6, characterized in that said multi-layered fabric structure comprises fabric layers being formed with pockets positioned between lower and upper ones of said fabric layers, said pockets being adapted for receiving one or more of said therapy magnets.

9. A head covering and hair restoration apparatus in accordance with claim 7, characterized in that said fabric layers are stitched or otherwise sewn together along a stitch line.

10. A head covering and hair restoration apparatus in accordance with claim 2, characterized in that said restoration insert is coupled to said head covering through permanent means.

11. A head covering and hair restoration apparatus in accordance with claim 2, characterized in that said restoration insert is secured to said head covering in a manner where said head covering is releasable from said restoration insert by said user.

12. A head covering and hair restoration apparatus in accordance with claim 2, characterized in that said restoration insert is separate and independent from said head covering.

13. A head covering and hair restoration apparatus in accordance with claim 2, characterized in that said restoration insert is adapted to be produced in selective sizes so as to fit scalps of users having differing remaining hairlines, head dimensions and other differing dimensions and properties.

14. A head covering and hair restoration apparatus in accordance with claim 2, characterized in that said therapy magnets have a magnetic field strength of 0.5 Gauss.

15. A head covering and hair restoration apparatus in accordance with claim 14, characterized in that said individual therapy magnets are of a disc-like configuration.

16. A head covering and hair restoration apparatus in accordance with claim 15, characterized in that said dimensions of said individual therapy magnets are a diameter of 0.75 inches, and a thickness of 0.125 inches.

17. A head covering and hair restoration apparatus in accordance with claim 16, characterized in that said therapy magnets of said magnet array are positioned so as to have a minimum spacing of 0.875 inches tip-to-tip.

18. A head covering and hair restoration apparatus in accordance with claim 16, characterized in that said magnets are spaced so that adjacent magnets are positioned no greater than 1.625 inches apart.

19. A head covering and hair restoration apparatus adapted for use by a user for curing hair loss and preventing further hair loss, said hair restoration apparatus comprising:
an electronic pulse generator connected to an electrical conducting cable extending within a shaped structure incorporated within and secured to said covering;
said restoration apparatus further comprising means connected to said conducting cable for supplying electrical power to said pulse generator and to said electrically conducting cable; and
said electrical pulse generator generating a pulsed electrostatic field between an interior of said head covering and the scalp of said user.

20. A head covering and hair restoration apparatus in accordance with claim 19, characterized in that said generator provides a pulsating electromagnetic field around said cable with an intensity of at least one Gauss in proximity to said cable.

21. A head covering and hair restoration apparatus in accordance with claim 20, characterized in that said sizing and configuration of said hair restoration apparatus is such that an intensity of said electromagnetic field generated by such pulse generator is substantially negligible at approximately 5 millimeters from a surface of said conducting cable.

22. A head covering and hair restoration apparatus in accordance with claim 21, characterized in that said electrical pulses comprise a duration of approximately 2 milliseconds, with a frequency of generation of approximately 50 pulses per second.

23. A head covering and hair restoration apparatus in accordance with claim 22, characterized in that a surface of said conducting cable which is abutting said user's skull is protected by plastic film or similar protective means.

24. A head covering and hair restoration apparatus in accordance with claim 1, characterized in that use of said hair restoration apparatus by said user is performed in combination with a nutritional regimen having a dosage of 5,000 I.U. of Vitamin D, once at night and once in the morning.

25. A head covering and hair restoration apparatus in accordance with claim 1, characterized in that said user uses said hair restoration apparatus in combination with a nutritional regimen comprising the ingestion of at least 100 grams of supplemental protein, taken in 4 doses throughout a daytime.

26. A head covering and hair restoration apparatus in accordance with claim 1, characterized in that said user wears said hair restoration apparatus for a period of 16 hours per day, with 8 hours of said 16-hour regiment preferably comprising a sleep state, and 8 hours of said 16-hour regiment preferably comprising daylight hours.
